(19) United States
(12) Patent Application Publication
(10) Pub. No.: US 2023/0225891 A1
(43) Pub. Date: Jul. 20, 2023

(54) MULTI-ROW DEPLOY ZONE CONSTRAINING DEVICES AND METHODS

(71) Applicant: W. L. Gore & Associates, Inc., Newark, DE (US)

(72) Inventor: Jerry J. Stastka, Flagstaff, AZ (US)

(21) Appl. No.: 18/009,125

(22) PCT Filed: Jun. 15, 2021
(86) PCT No.: PCT/US2021/037311

§ 371 Date: Dec. 8, 2022

Related U.S. Application Data
(60) Provisional application No. 63/039,834, filed on Jun. 16, 2020.

(51) Int. Cl. A61F 2/97; A61F 2/24; A61F 2/01; A61B 17/12; A61B 17/11; D04B 21/20; D04B 19/00

(52) U.S. Cl. CPC A61F 2/97; A61F 2/2439; A61F 2/011; A61B 17/12022; A61B 17/11; D04B 21/205; D04B 19/00; A61B 2017/00526

(57) ABSTRACT

A delivery system includes a catheter with a removable constraint. The removable constraint is a knit tubular construct. The removable constraint can be used, for example, to constrain a medical device to a delivery configuration. The removable constraint may include one or more fibers or strands arranged about the device to maintain the device in a constrained or delivery configuration.

### CROSS-REFERENCE TO RELATED APPLICATION

This application is a national phase application of PCT Application No. PCT/US2021/037311, internationally filed on Jun. 15, 2021, which claims the benefit of Provisional Application No. 63/039,834, filed Jun. 16, 2020, which are incorporated herein by reference in their entireties for all purposes.

### FIELD

The present disclosure relates generally to apparatuses, systems, and methods for delivery of implantable medical devices. More specifically, the disclosure relates to apparatuses, systems, and methods that include coverings for implantable medical devices during device delivery.

### BACKGROUND

Minimally invasive delivery techniques for implantable medical devices have a variety of advantages, such as reduced trauma, risk of infection, and recovery time. Examples of implantable medical devices include stents and stent-grafts utilized to radially support, treat and/or otherwise augment tubular passages in the body, including arteries, veins, airways, gastrointestinal tracts, and biliary tracts. Additional examples of implantable medical devices include prosthetic valves (e.g., prosthetic heart valves). Transcatheter delivery is a technique for delivering such implantable medical devices, where the medical device to be delivered begins in a diametrically compressed state for delivery and then is expanded (e.g., self-expanding or manually expandable) at a treatment site in the body of a patient.

Stents, stent-grafts, prosthetic valves, filters, and other implantables may be deployed by being plastically deformed (e.g., using an inflatable balloon) or permitted to self-expand and elastically recover from a collapsed or constrained, delivery diameter to an expanded, deployed diameter.

For example, U.S. Pat. No. 6,224,627, entitled “Remotely removable covering and support,” filed Jun. 15, 1998, describes, among other things, a thin tubular multiple filament (film or fiber) structure that can hold high internal pressures. When desired, an extension of the filaments can be pulled in any direction to unfurl the structure. The structure can be useful for self-expanding stent or stent graft delivery systems, balloon dilatation catheters, removable guide wire lumens for catheters, drug infusion or suction catheters, guide wire bundling casings, removable filters, removable wire insulation, removable packaging and other applications.

### SUMMARY

According to one example (“Example 1”), a knit tubular construct includes a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body.

According to another example (“Example 2”), further to the device of Example 1, corresponding knits within the respective knit rows of the at least two knit rows are configured to release substantially simultaneously as the at least two knit rows release progressively along the longitudinal length of the body.

According to another example (“Example 3”), further to Examples 1 or 2, a first fiber of the plurality of fibers defines a chain knit within one of the at least two knit rows.

According to another example (“Example 4”), further to Example 3, the plurality of knits forms a first knit row, a second knit row, and a third knit row, the first fiber interacting with each of the first, second, and third knit rows along the longitudinal length of the body.

According to another example (“Example 5”), further to Example 4, a second fiber of the plurality of fibers alternates between the first knit row and the second knit row along the longitudinal length of the body.

According to another example (“Example 6”), further to Examples 1 or 2, the plurality of fibers includes a first fiber, a second fiber, a third fiber, and a fourth fiber, wherein each of the first, second, third, and fourth fibers each define corresponding chain knits.

According to another example (“Example 7”), further to Example 6, the at least two knit rows includes a first knit row, a second knit row, a third knit row, and a fourth knit row, and wherein the first and second fibers define the corresponding chain knits in the first knit row and the third and fourth fibers define the corresponding chain knits in the third knit row.

According to another example (“Example 8”), further to Example 7, the fiber interacts with the second fiber in the first knit row, the third fiber in the second knit row, and the fourth fiber in the fourth knit row.

According to another example (“Example 9”), further to any of the preceding Examples, the plurality of knit rows are spaced laterally about a surface of the body.

According to another example (“Example 10”), further to any of the preceding Examples, the plurality of fibers each have a diameter of less than 0.0060″.

According to another example (“Example 11”), a medical device includes an expandable member configured to radially expand from a first diameter toward a second diameter, and a knit tubular construct configured to releasably radially constrain the expandable member, the knit tubular construct including a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body.

According to another example (“Example 12”), further to Example 11, the knit tubular construct includes a first knit row, a second knit row, and a third knit row, and the plurality of fibers of the knit tubular construct includes a cooperative fiber and an operative fiber, wherein the cooperative fiber forms at least a portion of a knit in the first, second, and third knit rows and the operative fiber form at least a portion of a knit in only two of the first, second, and third knit rows.

According to another example (“Example 13”), further to Example 12, the knit tubular construct includes at least two cooperative fibers.

According to another example (“Example 14”), further to Example 12, the knit tubular construct includes a first knit row, a second knit row, a third knit row and a fourth knit row, and wherein the plurality of fibers of the knit tubular construct includes a cooperative fiber, wherein the cooperative fiber forms at least a portion of a knit in at least three of the first, second, third, and fourth knit rows.

According to another example (“Example 15”), further to Example 14, the cooperative fiber forms a chain knit in at least one of the first, second, third, and fourth knit rows.

According to another example (“Example 16”), further to Example 15, the plurality of fibers includes a plurality of cooperative fibers.

According to another example (“Example 17”), further to Examples 12-16, the cooperative fiber is operable to increase resistance against deployment when the knit rows are not deployed substantially simultaneously.

According to another example (“Example 18”), further to Examples 12-17, the cooperative fiber is operable to increase a maximum constraining force of the knit tubular structure.

According to another example (“Example 19”), further to Examples 11-18, the at least two knit rows of the knit tubular construct are operable to release substantially simultaneously longitudinally along the body.

According to another example (“Example 20”), a method of deploying a medical device includes positioning an expandable member in a patient, wherein the expandable member is constrained by a knit tubular construct in a compressed configuration, wherein the knit tubular construct includes a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body, the plurality of fibers including deployment portions; retaining deployment portions of the plurality of fibers remote from the expandable medical device; and applying sufficient force to the deployment portions of the plurality of fibers to release at least a portion of the knit rows.

According to another example (“Example 21”), a method of manufacturing a medical device includes radially compressing an expandable member to a compressed profile; providing a knit tubular construct about the expandable member to constrain the expandable member in the compressed profile, the knit tubular construct including a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a delivery system having a removable constraint and an expandable member in accordance with an embodiment;

FIG. 2 is a removable constraint disposed about an expandable device in accordance with one embodiment;

FIG. 3 is a removable constraint with three knit rows and various knits in accordance with one embodiment;

FIG. 4 is a knit pattern implemented for a removable constraint as shown in FIG. 3, in accordance with one embodiment;

FIG. 5 is a removable constraint with four knit rows and various knits in accordance with on embodiment; and

FIG. 6 is a knit pattern implemented for a removable constraint as shown in FIG. 5, in accordance with one embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

For reference, the term “circumference” is not meant to require a circular cross-section, and is instead to be understood broadly to reference an outer surface, outer dimension, or perimeter of the removable constraint.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

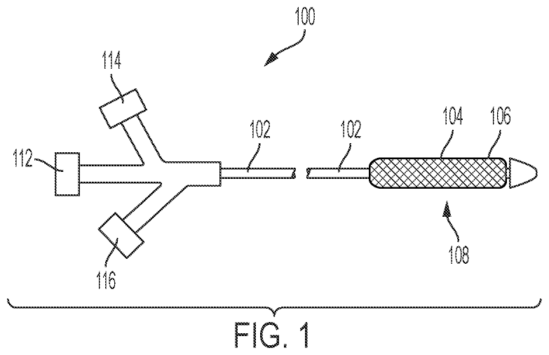

The system shown in FIG. 1 is provided as an example of the various features of the system and, although the combination of those illustrated features is clearly within the scope of invention, that example and its illustration is not meant to suggest the inventive concepts provided herein are limited from fewer features, additional features, or alternative features to one or more of those features shown in the figures. FIG. 1 is a plan view of a delivery system 100 including a catheter 102 with a removable constraint 104, according to some embodiments. In some embodiments, the removable constraint 104 is a knit tubular construct. As shown in FIG. 1, the removable constraint 104 is configured to constrain an implantable medical device 106 to a delivery configuration. The removable constraint 104 may include one or more fibers or strands 108 arranged about the device 106 to maintain the device 106 and the removable constraint 104 in a constrained or delivery configuration. The catheter 102 may include various ports, for example, a first port 112, a second port 114, and a third port 116. One or more of the ports 112, 114, 116 may be configured to provide access to one or more features (e.g., lumens) or to operate one or more functions (e.g., constraint release) as desired.

The removable constraint 104 is arranged along a length of the device 106. The removable constraint 104 is also circumferentially arranged about the device 106 and may substantially cover the device 106 for delivery. The one or more strands 108 may be arranged within a lumen (not shown) of the catheter 102 and extend toward a proximal end of the catheter 102, which may, in turn, be arranged external to a patient during delivery of the device 106. The one or more strands 108 may include a proximal end 110 that a user may tension in order to release the removable constraint 104 and deploy the device 106. For example, the one or more strands may be accessible through one or more of the ports 112, 114, 116.

In certain instances, the one or more strands 108 release such that interlocking portions (e.g., overlapping fibers or knits) sequentially and progressively release along the length of the device 106. As is explained in greater detail below, the removable constraint 104 is formed by interlocking together the one or more strands 108 extending around the device 106. The one or more strands 108 may form knit rows 130 where the one or more strands 108 interact with each other. The configuration of the knit rows 130 and the one or more strands 108 forming the knit rows 130 provide certain properties to facilitate constraint of the device 106 in the constrained configuration and release or deployment of the device from the removable constraint 104 to the deployed configuration. In some embodiments, the device 106 may be a stent, stent-graft, a balloon, prosthetic valve, filter, anastomosis device, occluder or a similar device.

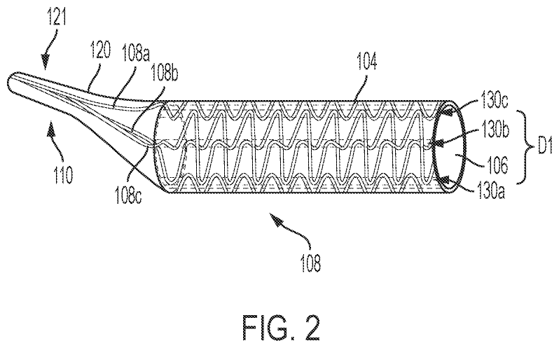

FIG. 2 is a side view of the device 106 including the removable constraint 104, in accordance with an embodiment. The device 106 is configured to be transitioned from a delivery diameter D1 to a deployed diameter D2 (not shown) that is larger than the delivery diameter D1. In various examples, the removable constraint 104 is arranged about the device 106 at the delivery diameter D1. When the removable constraint 104 is removed from the device 106, the device is expandable to a deployed diameter D2 (e.g., via self-expansion and/or forced expansion, such as balloon expansion). The deployed diameter D2 is greater than the delivery diameter D1. In some embodiments, the deployed diameter D2 is the diameter of the device 106 when unconstrained. In other embodiments, the deployed diameter D2 is the diameter of the device 106 once the device 106 has been delivered to a target site and has engaged with the lumen wall at the target site.

The device 106 may have a desired deployed diameter D2 from about 5 mm-15 mm, or 6 mm-9 mm, or 6 mm-12 mm, for example, and a delivery diameter D1 that is less than the deployed diameter D2. For example, in some instances, a ratio of the delivery diameter D1 of the device 106 to the deployed diameter D2 (not shown) of the device 106 is less than about 0.3, less than about 0.29, less than about 0.28, less than about 0.27, or less than about 0.26.

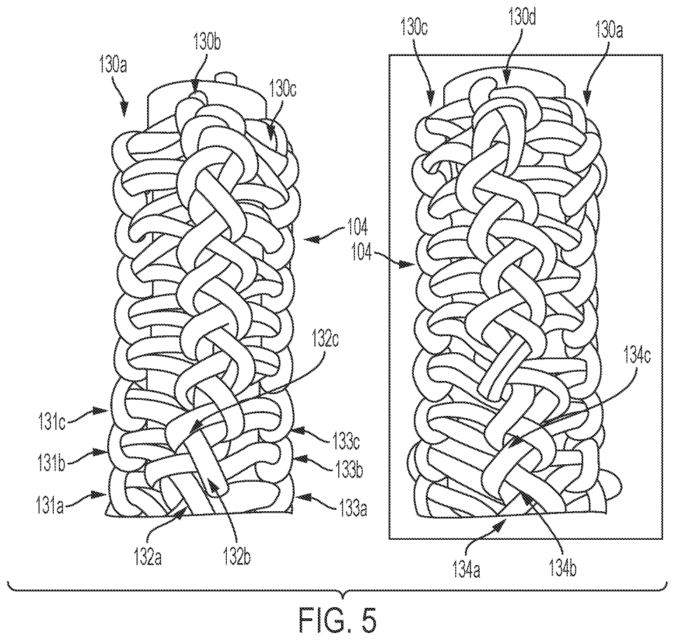

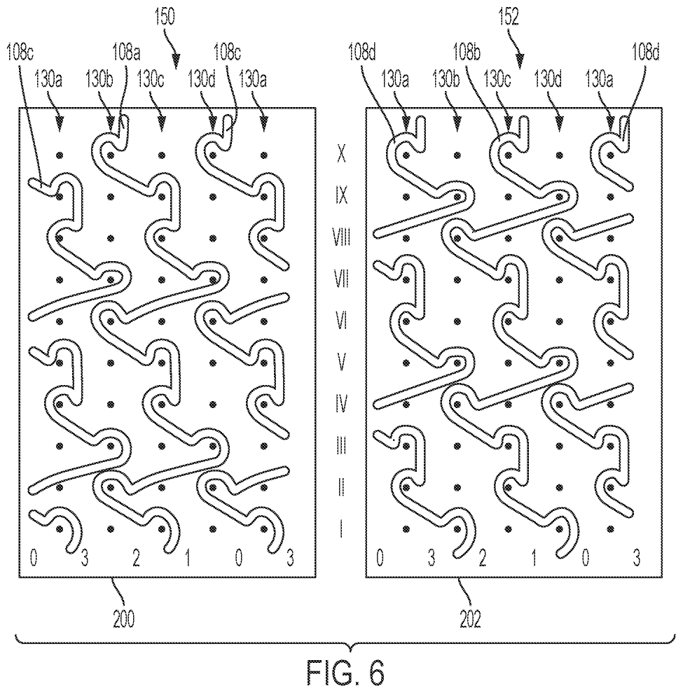

As shown in FIG. 2, the removable constraint 104 includes at least two strands 108 interlocking in the form of a warp knit. For example, the removable constraint 104 may include a first interlocking strand 108a and a second interlocking strand 108b. Portions of the first and/or the second interlocking strand(s) 108a, 108b may operate, for example, as deployment portions 120 configured to release the removable constraint 104 and release the device 106 from the delivery diameter D1 to the deployed diameter D2 in response to a deployment force applied to the first deployment line portion. The removable constraint 104 may also include a third interlocking strand 108c and a fourth interlocking strand 108d (for example, as seen in FIGS. 5 and 6). The third and/or the fourth interlocking strand(s) 108c, 108d may likewise operate, for example, as the deployment portion 120. It is within the scope of this disclosure to form a removable constraint with two, three, four, five, six, seven, eight, nine, any even number of interlocking strands or any odd number of interlocking strands. In one embodiment, the deployment portions 120 are coupled together to form a unitary deployment segment 121. In some embodiments the deployment portions 120, which may combine to define the unitary deployment segment 121, includes the proximal end 110 of the one or more strands 108.

In some examples, the device 106 is self-biased toward the deployed diameter to exert an outward radial force when constrained at the delivery diameter D1. In some examples, an expansion force (e.g., a balloon) may additionally or alternatively be applied to the device 106 such that expansion force acting on the device 106 applies a radial force to the constraint. The constraint 104 may also be released prior to imparting an expansion force on the device 106. Where the device 106 is self-expanding, the radial force generally refers to the force caused by the device 106 acting on the removable constraint 104 at any point during deployment of the device 106.

As discussed above, the interlocking strands 108 are adapted to be removed with a deployment force applied to the deployment portion 120. Low deployment force may be preferable to permit the deconstruction of the removable constraint 106 such that the device may be deployed without having to apply large forces to the deployment portions 120, which may inadvertently result in displacement of the device 106 from the target location. In some instances, a lower profile may be achieved for a given deployment force by utilizing a plurality (e.g., 2, 3, 4) of knit rows with smaller strands as compared to a similar deployment force (or constraining force) from a larger strand single knit row deployment. Strands can include a diameter from about 0.0010″ to about 0.0100″. For example, the interlocking strands 108 used to form the removable constraint 104 have a diameter of about 0.0038″ to about 0.0054″. To maintain a lower profile, the interlocking strands 108 may each have a diameter of less than 0.0060″. For an even lower profile, the interlocking strands 108 may each have a diameter of less than 0.0040″. Because the interlocking strands 108 are engaged as discussed herein, the profile may be minimized while still maintaining sufficient constraining force (and appropriate deployment force). In some instances, the ratio of this radial force of the device 106 to the deployment force applied to the deployment portions 120 is less than about 500:1. In other instances, the ratio of this radial force of the device 106 to the deployment force applied to the deployment portions 120 is less than about 475. In addition, the ratio of this radial force of the device 106 to the deployment force applied to the deployment portions 120 may be less than about 450. In addition, the ratio of this radial force of the device 106 to the deployment force applied to the deployment portions 120 is less than about 425 in other instances. Further, the ratio of the radial force to the deployment force may be between about 10, 20, 30, 40, 50, 100, 200, 300, 400 (or any number in between) and about 500, between about 10, 20, 30, 40, 50, 100, 200, 300, 400 (or any number in between) and about 475, between about 10, 20, 30, 40, 50, 100, 200, 300, 400 (or any number in between) and about 450, or between about 10, 20, 30, 40, 50, 100, 200, 300, 400 (or any number in between) and about 425, for example.

The one or more strands 108, including the interlocking strands 108a, 108b, 108c, 108d in some embodiments, may be formed of various materials, including, for example, polytetrafluoroethylene (PTFE), expanded polytetrafluoroethylene (ePTFE), polyester, polyurethane, fluoropolymers, such as perfluoroelastomers and the like, silicones, urethanes, aramid fibers, and combinations thereof. Other embodiments for strands 108 can include high strength polymer fibers such as ultra-high molecular weight polyethylene fibers (e.g., Spectra®, Dyneema Purity®, etc.) or aramid fibers (e.g., Technora®, etc.).

The various strands 108 may be selected to have specific properties such as strand thickness, strand denier, strand coefficient of friction, strand material, strand treatments, strand coatings, and strand stiffness. Similar to the strand thickness, use of differing strand materials for the strands 108 may increase or decrease friction between the first and second interlocking strands 108 to help maintain or optimize the device 106 in the delivery configuration. Each of the various strands may be selected to include the same strand properties or different strand properties based on the application in which the removable constraint will be used. It is recognized that the properties of the strands 108 may also be altered by treatments, configurations, and alterations, in addition to material selection. For example, the strands may include fillers or core materials, may be surface treated by etching, vapor deposition, or coronal or other plasma treatment, among other treatment types, including being coated with suitable coating materials.

Referring to FIG. 2 which provides an exemplary embodiment of a removable constraint 104 constraining an expandable device 106, the removable constraint 104 includes knit rows 130 formed by the knitting of the various strands 108. Any number of knit rows 130 may be implemented in connection with the removable constraint 104. For example, the removable constraint 104 may include a first knit row 130a, a second knit row 130b, and a third knit row 130c. As seen in FIG. 2, a knit pattern is overlaid on a removable constraint 104 for reference. Each of the knit rows 130a, 130b, 130c shown in FIG. 2 act as a release zone at which the removable constraint 104 is operable to unravel to release or deploy the expandable device 106. The knit rows 130 may be defined along at least a portion of a longitudinal length of the removable constraint 104. In some embodiments the knit rows 130 are coextensive along the longitudinal length of the removable constraint 104. The knit rows 130 may also be circumferentially spaced from each other along the outer dimension or circumference of the removable constraint 104. The knit rows 130 may be spaced approximately equidistant about the circumference of the removable constraint 104, or they may be offset as desired.

The circumferential distance between the knit rows 130 may be described in terms of arc angles in those embodiments where the removable constraint surrounds the device 106. In some embodiments, the knit rows 130 may be disposed on a first face of the removable constraint 104 such that all knit rows 130 are positioned within about 180 degrees of the removable constraint 104. The knit rows 130 may be spaced relative to each other about the circumference of the removable constraint 104 from about 10 degrees to about 180 degrees, from about 20 degrees to about 30 degrees, from about 30 degrees to about 45 degrees, from about 45 degrees to about 60 degrees, from about 60 degrees to about 75 degrees, from about 75 degrees to about 90 degrees, from about 90 degrees to about 105 degrees, from about 105 degrees to about 120 degrees, from about 120 degrees to about 135 degrees, from about 135 degrees to about 145 degrees, from about 145 degrees to about 160 degrees, from about 160 degrees to about 180 degrees. For example, the knit rows can be spaced approximately 180 degrees apart from one another, approximately 90 degrees apart from one another, approximately 60 degrees apart from one another, or any other distance as desired.

Referring to FIG. 2, the removable constraint 104 includes three knit rows 130a, 130b, 130c, according to some examples. The knit rows 130a, 130b, 130c are formed by the interlocking of the various strands 108a, 108b, 108c previously discussed. It is understood that the removable constraint 104 is not limited to only three knit rows, but any number of knit rows may be implemented, including a fourth knit, a fifth knit row, or any number of knit rows. In some embodiments, the number of knit rows corresponds to the number of strands implemented in forming the removable constraint 104.

The removable constraint 104 may include deployment portions 120 that together form the unitary deployment segment 121. The unitary deployment segment 121 is configured to deploy the device 106 by disengaging the removable constraint 104 from the device 106. This may occur via an unravelling of the knit rows 130 and consequently portions of the body of the removable constraint 104. In one embodiment, the first deployment line 108a extends from the first knit row 130a and is engaged with (e.g., forms a portion of) the first knit row 130a such that the first deployment portion 120 is operable to disengage or unravel at least a first portion of the first knit row 130a. The unitary deployment segment 121 includes portions of each of the strands 108 comprising the knit rows 130. Each of the knit rows 130 is operably to sequentially unravel as the unitary deployment segment 121 is engaged.

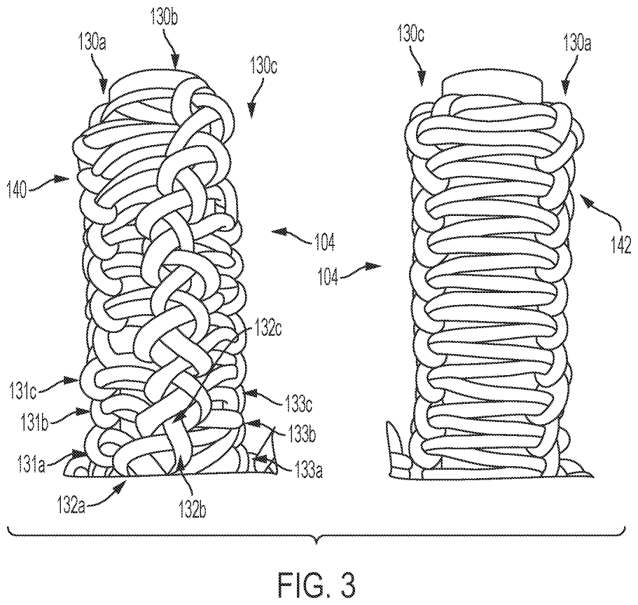

An example is shown in FIG. 3 in which a removable constraint 104 has a first knit row 130a, a second knit row 130b, and a third knit row 130c. As can be seen in FIG. 3, the first knit row 130a and the second knit row 130b are positioned spaced from each other about 90 degrees along the outer dimension of the removable constraint 104. The second knit row 130b and the third knit row 130c are likewise spaced from each other about 90 degrees along the outer dimension of the removable constraint 104. Consequently, the first knit row 130a and the third knit row 130c are spaced from each other about 180 degree along the outer dimension of the removable constraint 104. As can be seen in FIG. 3, all three of the knit rows 130 may be positioned substantially on a front face 140 of the removable constraint 104, whereas the knit rows are not positioned on a back face 142. The spacing of the knit rows 130 may be varied as previously discussed.

As can be seen in FIG. 3, various knits may also be positioned within each of the knit rows 130 along the longitudinal length of the removable constraint 104. For example, the first knit row 130a includes a first knit 131a, a second knit 131b, a third knit 131c, and so forth. The second knit row 130b also includes a first knit 132a, a second knit 132b, a third knit 132c, and so forth. The third knit row 130c also includes a first knit 133a, a second knit 133b, a third knit 133c, and so forth. Each of the knits 131, 132, 133 includes interwoven portions of at least one of the strands 108.

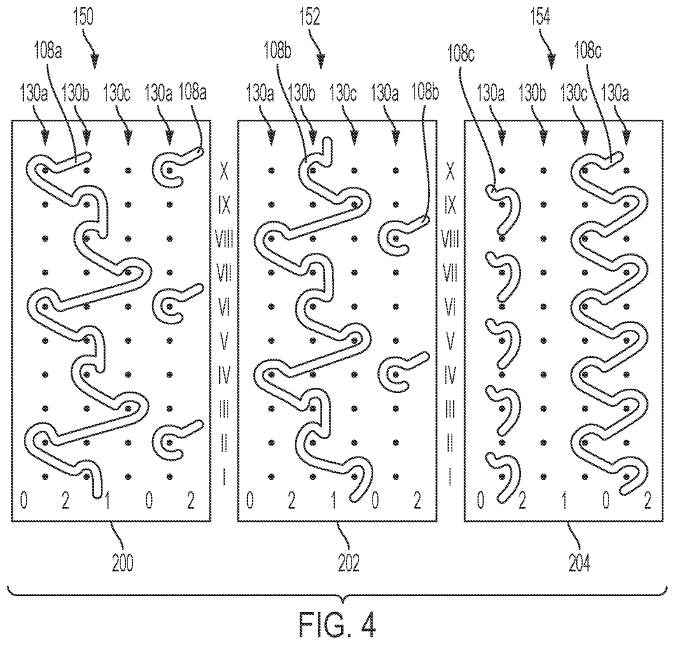

Referring to FIG. 4, an exemplary embodiment of knit pattern is provided. The knit pattern of FIG. 4 includes a first strand 108a, a second strand 108b, and a third strand 108c. The first, second, and third strands 108 each include a separate pattern represented by a first pattern 150, a second pattern 152, and a third pattern 154, respectively. In some embodiments, each pattern may be implemented on a knitting machine (e.g., circular warp knitting machines, straight bar, flat bar, Raschel, Milanese, tricot, and so forth). In order to implement certain patterns, the knitting machine may include a plurality of bars. For example, the first strand 108a may correspond to a first bar 200, the second strand 108b may correspond to a second bar 202, and the third strand 108c may correspond to a third bar 204, wherein each bar implements a different pattern or patterns. In some embodiments the different patterns may include phase shifts relative to one another.

Although the removable constraint 104 is not limited to a specific process of manufacture, the following knit structure is provided with relation to a knitting machine and being implemented on a knitting machine (e.g., circular warp knitting machines, straight bar, flat bar, Raschel, Milanese, and tricot). The following knit structure is a four-course repeat. The first bar 200 in this example includes the following repeated knit structure: 1-2/0-2/0-1/2-1. When the knit structure repeats, a chain or pillar knit is formed between the first and the last course in the knit structure. It is noted that because a tubular structure is being formed, the knit course 0-2 and then 0-1 is such that the first strand 108a extends across 2 and 1 between 0-2 and 0-1. It is understood that two needle bars (not shown) may be implemented to form the tubular structure. The second bar 202 in this example includes the following repeated knit structure: 0-1/2-1/1-2/0-2. When the four-course structure repeats, similar to the first bar 200, the knit course 0-2 and then 0-1 is such that the second strand 108b extends across 2 and 1 between 0-2 and 0-1. In this example, the first strand 108a and the second strand 108b include similar knit structures out of phase relative to each other. The third bar 204 in this example includes the following repeated knit structure: 2-0/1-0/2-0/1-0. The third strand 108c alternates between two positions in the knit structure. In other terms, the first and the second strands 108a, 108b may be described as having chain or pillar knits in a common knit row (e.g., the second knit row 130b) and then alternate between the two adjacent knit rows (that is, adjacent to the common knit row) across the common knit row (e.g., the first and third knit rows 130a, 130c across the second knit row 130b). The third strand 108c alternates between two knit rows (e.g., between the first and third knit rows 130a, 130c, although not across the second knit row 130b, but directly between each other because the knit structure is tubular). Thus in this example, the first and the second strands 108a, 108b may be considered cooperative strands as the strands interact with at least three of the knit rows 130 and the third strand 108c may be considered the operative strand as it interacts with less than three of the knit rows. It is noted that the each of the knits in this knit structure is an open knit. The open knit structure facilitates unraveling of the removable constraint 104, and consequently deployment of the expandable device 106.

It is noted that the embodiment disclosed above may be implemented with more than three strands, including embodiments having a number of strands that are multiples of three (e.g., six strands, nine strands, twelve strands, and so forth). In those embodiments, instead of the first and third knit rows 130a, 130c looping back and connecting to each other, the third knit row 130c may instead extend to a fourth knit row (not shown), where the fourth knit row begins a repeat of the disclosed pattern.

Referring now to FIG. 5, an example is provided in which a removable constraint 104 has a first knit row 130a, a second knit row 130b, a third knit row 130c, and a fourth knit row 130d. As can be seen in FIG. 5, each of the knit rows 130 are evenly spaced. Specifically, the first knit row 130a and the second knit row 130b are positioned spaced from each other about 90 degrees along the outer dimension of the removable constraint 104. The second knit row 130b and the third knit row 130c are likewise spaced from each other about 90 degrees along the outer dimension of the removable constraint 104. Consequently, the first knit row 130a and the third knit row 130c are spaced from each other about 180 degree along the outer dimension of the removable constraint 104. The third knit row 130c and the fourth knit row 103d are spaced from each other about 90 degrees along the outer dimension of the removable constraint 104. Consequently, the second knit row 130b and the fourth knit row 130d are spaced from each other about 180 degree along the outer dimension of the removable constraint 104. The spacing of the knit rows 130 may be varied such that the knit rows are not evenly spaced, as previously discussed.

As can be seen in FIG. 5, various knits may also be positioned within each of the knit rows 130 along the longitudinal length of the removable constraint 104. For example, the first knit row 130a includes a first knit 131a, a second knit 131b, a third knit 131c, and so forth. The second knit row 130b also includes a first knit 132a, a second knit 132b, a third knit 132c, and so forth. The third knit row 130c also includes a first knit 133a, a second knit 133b, a third knit 133c, and so forth. The fourth knit row 130d also includes a first knit 134a, a second knit 134b, a third knit 134c, and so forth. Each of the knits 131, 132, 133, 134 includes interwoven portions of at least one of the strands 108.

Referring to FIG. 6, an exemplary embodiment of knit pattern is provided. The knit pattern of FIG. 6 includes a first strand 108a, a second strand 108b, a third strand 108c, and a fourth strand 108d. In this knit pattern, the first and third strands 108, 108c have separate knit patterns from the second and fourth strands 108b, 108d. The first knit pattern 150 is shown on the first bar 200 and the second knit pattern 152 is shown on the second bar 202. Because the first and third strands 108a, 108c include the same first pattern 200, they may be woven on the same bar at the same time, wherein the strands are laterally spaced from each other. The same is applicable to the second and forth strands 108b, 108d. In some embodiments, each pattern may be implemented on a knitting machine (e.g., tricot, Milanese, Raschel, and so forth). It will be noted that the first and second strands 108a, 108b have a similar overall pattern, but include a phase shift relative to each other, in this embodiment, and accordingly are implemented on separate bars. The same is true for the third and fourth strands 108c, 108d, in this embodiment.

Although the removable constraint 104 is not limited to a specific process of manufacture, the following knit structure is provided with relation to a knitting machine and being implemented on a knitting machine. The following knit structure is a four-course repeat. The first bar 200 in this example includes the following repeated knit structure for the first strand 108a and the third strand 108c, respectively: 1-2/3-2/0-1/2-1 and 3-0/1-0/2-3/0-3. When the knit structure repeats, a chain or pillar knit is formed between the last course of the pattern and the first course of the repeated pattern in the knit structure. It is noted that because a tubular structure is being formed, the knit may extend to form a tubular structure. This may be accomplished, in some embodiments, by implementing two needle bars (not shown). The second bar 202 in this example includes the following repeated knit structure for the second strand 108b and the fourth strand 108d, respectively: 0-1/2-1/1-2/3-2 and 2-3/0-3/3-0/1-0. As can be seen, a chain or pillar knit is included in both the second and fourth strands 108b, 108d. In this example, the first strand 108a and the second strand 108b include similar knit structures out of phase relative to each other. Because the first and second strands 108a, 108b include similar knit patterns out of phase with each other, the first and the second strands 108a, 108b may be described as having chain or pillar knits in a common knit row (e.g., the second knit row 130b) and then alternate between the two adjacent knit rows (that is, adjacent to the common knit row) across the common knit row (e.g., the first and third knit rows 130a, 130c across the second knit row 130b). Similarly, because the third and fourth strands 108c, 108d include similar knit patterns out of phase with each other, the third and the fourth strands 108c, 108d may be described as having chain or pillar knits in a common knit row (e.g., the fourth knit row 130d) and then alternate between the two adjacent knit rows (that is, adjacent to the common knit row) across the common knit row (e.g., the first and third knit rows 130a, 130c across the fourth knit row 130d). Thus in this example, the first, second, third, and fourth strands 108a, 108b, 108c, 108d may all be considered cooperative strands as the strands interact with both knit rows adjacent to the knit row in which the chain or pillar knit is formed. It is noted that the each of the knits in this knit structure is an open knit. The open knit structure facilitates unraveling of the removable constraint 104, and consequently deployment of the expandable device 106.

Referring to FIG. 6, each of the knit rows 130a, 130b, 130c, 130d include warp knits. It is noted that the removable constraint 104 includes chain or pillar knits in two of the four knit rows (e.g., the first and third knit rows 130a, 130c). Because the chain or pillar knits are included in two of the four knit rows, the two strands that have chain or pillar knits in the same knit row include the same pattern, only the patterns are out of phase with each other by two courses (e.g. the first and the second strand 108a, 108b include chain or pillar knits in the third knit row 103c have the same pattern out of phase with each other by two courses). It is noted that the strands that form chain or pillar knits in the same knit row only interact with each other to form a knit in the knit row in which they form the chain or pillar knit (e.g., the first and the second strand 108a, 108b only interact to form a knit in the third knit row 103c).

As previously discussed, the embodiment shown in FIG. 6 includes two strands being woven on a single bar (e.g., the first and third strand 108a, 108c being woven on a first bar 200). The two strands may be implemented on a single bar because the strands are woven using the same pattern but offset in different knit rows. These strands may be considered to be in phase with each other. Because the two strands are in phase with each other and the other two strands are out of phase by two courses, chain or pillar knits are formed in the removable constraint between every second course (e.g., between courses II and III and between courses IV and V). This is a result of the two bars 150, 152 including knit patterns that are out-of-phase by two courses. For those knit rows that include chain or pillar knits, the chain or pillar knit occurs in that knit row between every fourth course (e.g., the third knit row 103b has a chain or pillar knit between courses IV and V and between courses VIII and IX). It is also noted that in this embodiment, each of the strands interacts to form a knit with each of the remaining strands (e.g., the first strand 108a interacts with the second strand 108b to form a knit, the third strand 108c to form a knit, and the fourth strand 108d to form a knit). Because the various strands interact with each other and in various knit rows, the constraining force of the removable constraint 104 is increased. This also reduces uncontrolled, spontaneous, or accelerated deployment from occurring because each of the strands are actuated substantially simultaneously to deploy the removable constraint 104 in a sequential pattern as dictated by the knit pattern. Because each strand can include a different knit pattern or be out of phase with each other, the slack from each strand may not be equal after knits from the same course are deployed which may also reduce uncontrolled, spontaneous, or accelerated deployment from occurring.

The embodiment disclosed above may be implemented with more than four strands, including embodiments having a number of strands that are multiples of two (e.g., two strands, six strands, eight strands, ten strands, and so forth).

Referring again to FIG. 2, the removable constraint 104 may include a unitary deployment segment 121 which includes extensions of or free ends of the each of the strands that form each of the knit rows 130. When the unitary deployment segment 121 is engaged or tensioned such that free portions of each of the strands 108 advances away from the removable constraint 104, the first knit (e.g., first knits 131a, 132a, 133a, 134a) of each knit row (e.g., first, second, third and fourth knit rows 130a, 130b, 103c, 130d) unravel. As the unitary deployment segment 121 continues to be tensioned and translated away from the removable constraint 104, the second knits (e.g., 131b, 132b, 133b, 134b) of each knit row unravels. This occurs until each of the knit rows has partially or fully unraveled.

In some embodiments, the interwoven strands 108 are knit such that the knit rows 130 are deployed substantially simultaneously in order to facilitate the unraveling of the knit rows. Because the strands 108 are all interwoven, when any one of the knit rows is advanced or unraveled at a different rate than the other knit rows, the strands forming the other knit rows may interfere with the proper unraveling of the former knit row. This occurs by the binding or restriction of the strand 108 at one knit row until the other knit row has been sufficiently advanced to release the strand from the other knit row. Because all of the strands 108 may be interwoven, this restriction of deployment may occur when any one of the knit rows 130 are unraveled disproportionately relative to each other.

For example, if the first strand 108a is tensioned such that the first knit 131a of the first knit row 130a is unraveled and the first strand 108a continues to be tensioned and the corresponding first knits (e.g., first knit 132a of the second knit row 130b) has not been unraveled, the strands from the corresponding first knits interacting with the second knit 131b of the first knit row may interfere with the unraveling of the second knit 131b. In this example, the first strand 108a may be interwoven with the remaining strands in any one of the knit rows 130 such that the first strand 108a is unable to advance further until the other strands forming the other knits constraining first strand 108a are released from the other knits via the release or unraveling of the other knits. However, if the other knits are unraveled, the first strand 108a may be freed from the other strands at the position at which it was restricted such that tension across the first strand 108a may initiate deployment of a subsequent knit, which is then unraveled. In this manner, uneven deployment of the various knit rows 130 is restricted by the interweaving of the various strands 108 within each knit row 130.

In some embodiments, the corresponding knits each of the knit rows 130 must be deployed before the subsequent knit in the knit row can be sequentially deployed. Because the cooperative strands interact with at least three knit rows, the cooperative strands may provide increased stability, increased constraining force, and increased resistance against accelerated deployment with relation to an expandable device and deployment of the knit rows 130 of a removable constraint 104. The cooperative strands may also provide increased maximum constraining force for the removable constraint 104 based on the interactions described herein. In other embodiments, the strands 108 are interwoven such that the subsequent knits may be deployed when the corresponding knits of the other knit row are not deployed. In yet another embodiment, the strands 108 are interwoven such that a subsequent knit (e.g., second knit 131b) in a knit row may deploy when a corresponding knit (e.g., corresponding first knit 133a) is undeployed; however, a knit subsequent to the subsequent knit (e.g., third knit 131c) may be restricted when the corresponding knit is undeployed. The pattern for interweaving the various strands may be altered to provide various interactions between the knit rows for restricting unraveling. For instance, a knit row may advance or be unraveled at two knits, three knits, four knits, or five knits beyond the unravelling of the corresponding intact knits of the other knit row based on the weave or knit pattern. By varying how far a knit row may advance past the other knit row in unraveling, the precision of deployment may be varied when delivering and deploying a device 106.

Turning now to a discussion of the methods for making and using a removable constraint, the method of deploying a device with the disclosed removable constraint is provided. As previously discussed, a medical device may include an expandable device capable of expanding and contracting to various diameters, including a first constrained diameter D1 and a second expanded diameter D2. The expandable device may be maintained in a constrained configuration by a removable constraint, the removable constraint comprising a plurality of strands interwoven to form a first release zone and a second release zone, each comprising knit rows with a plurality of knits. In some embodiments, at least one deployment line extends from each of the release zones.

The method of deploying a medical device may include delivering the device to the treatment site intravenously. The expandable medical device is positioned in a patient, wherein the expandable medical device is constrained by a removable constraint in a compressed configuration. The knit rows configured to disengage the removable constraint from the expandable medical device via the unitary deployment segment. A user may retain the unitary deployment segment (e.g., comprising free ends of the strands forming the removable constraint) remote from the expandable medical device, i.e., outside of the intravenous access site. The user may then apply sufficient force to the unitary deployment segment to release the knit rows. As the knit rows are released, the medical device may be released from the removable constraint and deploy within the anatomy of the patient. Thus, as the knit rows release, the removable constraint is at least partially deconstructed and the expandable device is able to expand from the constrained diameter to a deployed diameter.

In some embodiments, the method includes simultaneously applying sufficient force to the free ends of the strands, or applying such force in relatively close temporal sequence. As discussed above, this step may be important when the plurality of strands are interwoven, such that the knits interfere with the release or deployment of the corresponding knits. As the free ends of the strands are activated, they may be translated away from the delivery site. The plurality of lines may be removed via the catheter.

The disclosure also relates to a method of manufacturing an expandable medical device. The method may include compressing an expandable member radially inward to a first compressed diameter. A plurality of strands including may be interwoven to form a removable constraint. The removable constraint may be interwoven such that the strands are form at least three knit rows. The method may include providing free ends of the strands such that at least a portion of each of the strands extends away from the removable constraint. The free ends may be operable to deconstruct the removable constraint when deployed substantially simultaneously. The method may also include coupling the free ends such that they form a unitary deployment segment.

In some embodiments, the method of manufacturing includes performing the step of compressing the expandable member simultaneously with the step of forming the removable constraint such that the plurality of strands provide a compressive force to the expandable member as the plurality of strands are interwoven about the expandable member.

In some embodiments, the covering member may be woven on a mandrel. Once the covering member is woven, and in some embodiments partially deployed, the covering member may be removed from the mandrel and applied over a radially compressed implantable medical device.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A knit tubular construct comprising:
a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body.

2. The knit tubular construct of claim 1, wherein corresponding knits within the respective knit rows of the at least two knit rows are configured to release substantially simultaneously as the at least two knit rows release progressively along the longitudinal length of the body.

3. The knit tubular construct of either claim 1, wherein a first fiber of the plurality of fibers defines a chain knit within one of the at least two knit rows.

4. The knit tubular construct of claim 3, wherein the plurality of knits forms a first knit row, a second knit row, and a third knit row, the first fiber interacting with each of the first, second, and third knit rows along the longitudinal length of the body.

5. The knit tubular construct of claim 4, wherein a second fiber of the plurality of fibers alternates between the first knit row and the second knit row along the longitudinal length of the body.

6. The knit tubular construct of claim 1, wherein the plurality of fibers includes a first fiber, a second fiber, a third fiber, and a fourth fiber, wherein each of the first, second, third, and fourth fibers each define corresponding chain knits.

7. The knit tubular structure of claim 6, wherein the at least two knit rows includes a first knit row, a second knit row, a third knit row, and a fourth knit row, and wherein the first and second fibers define the corresponding chain knits in the first knit row and the third and fourth fibers define the corresponding chain knits in the third knit row.

8. The knit tubular construct of claim 7, wherein the fiber interacts with the second fiber in the first knit row, the third fiber in the second knit row, and the fourth fiber in the fourth knit row.

9. The knit tubular construct of claim 1, wherein the plurality of knit rows are spaced laterally about a surface of the body.

10. The knit tubular construct of claim 1, wherein the plurality of fibers each have a diameter of less than 0.0060″.

11. A medical device comprising:
an expandable member configured to radially expand from a first diameter toward a second diameter; and
a knit tubular construct configured to releasably radially constrain the expandable member, the knit tubular construct including a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body.

12. The medical device of claim 11, wherein the knit tubular construct includes a first knit row, a second knit row, and a third knit row, and wherein the plurality of fibers of the knit tubular construct includes a cooperative fiber and an operative fiber, wherein the cooperative fiber forms at least a portion of a knit in the first, second, and third knit rows and the operative fiber form at least a portion of a knit in only two of the first, second, and third knit rows.

13. The medical device of claim 12, wherein the knit tubular construct includes at least two cooperative fibers.

14. The medical device of claim 12, wherein the knit tubular construct includes a first knit row, a second knit row, a third knit row and a fourth knit row, and wherein the plurality of fibers of the knit tubular construct includes a cooperative fiber, wherein the cooperative fiber forms at least a portion of a knit in at least three of the first, second, third, and fourth knit rows.

15. The medical device of claim 14, wherein the cooperative fiber forms a chain knit in at least one of the first, second, third, and fourth knit rows.

16. The medical device of claim 15, wherein the plurality of fibers includes a plurality of cooperative fibers.

17. The medical device of claim 12, wherein the cooperative fiber is operable to increase resistance against deployment when the knit rows are not deployed substantially simultaneously.

18. The medical device of claim 12, wherein the cooperative fiber is operable to increase a maximum constraining force of the knit tubular structure.

19. The medical device of claim 11, wherein the at least two knit rows of the knit tubular construct are operable to release substantially simultaneously longitudinally along the body.

20. A method of deploying a medical device, comprising:
positioning an expandable member in a patient, wherein the expandable member is constrained by a knit tubular construct in a compressed configuration, wherein the knit tubular construct includes a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body, the plurality of fibers including deployment portions;
retaining deployment portions of the plurality of fibers remote from the expandable medical device; and
applying sufficient force to the deployment portions of the plurality of fibers to release at least a portion of the knit rows.

21. A method of manufacturing a medical device comprising:
radially compressing an expandable member to a compressed profile; and
providing a knit tubular construct about the expandable member to constrain the expandable member in the compressed profile, the knit tubular construct including a plurality of fibers forming a body having a longitudinal length, the plurality of fibers defining a plurality of knits, the plurality of knits forming at least two knit rows extending longitudinally along the body, the at least two knits rows configured to release progressively along at least a portion of the longitudinal length of the body.
